(19) United States
(12) Patent Application Publication
(10) Pub. No.: US 2023/0132821 A1
(43) Pub. Date: May 4, 2023

(54) TOTAL KNEE JOINT PROSTHESIS FOR REVISION AFTER A UNICOMPARTMENTAL KNEE ARTHROPLASTY

(71) Applicant: Beijing LiDaKang Technology Co.,Ltd., Beijing (CN)

(72) Inventors: Yapeng ZHANG, Beijing (CN); Shuqin MA, Beijing (CN); Jingliang JIA, Beijing (CN)

(21) Appl. No.: 17/974,973

(22) Filed: Oct. 27, 2022

(30) Foreign Application Priority Data
Nov. 1, 2021 (CN) 202111283975.6

(51) Int. Cl. A61F 2/38; A61B 17/17

(52) U.S. Cl. CPC A61F 2/389; A61B 17/1764; A61F 2/46

(57) ABSTRACT

The present application relates to the technical field of biomaterials for implant and intervention, and particularly to a total knee joint prosthesis for revision after a unicompartmental knee arthroplasty including a tibial plateau and a tibial pad mounted on the tibial plateau; the tibial plateau includes an installation platform and an installation column fixedly connect to a side of the installation platform; a filling block is provided on a side of the installation platform towards the installation column; and the filling block is configured to be placed at a side of the human tibia where a unicompartmental knee arthroplasty is performed.

### CROSS-REFERENCE TO RELATED APPLICATION

The present application is based on and claims the priority benefits of China application No. 202111283975.6, filed on Nov. 1, 2021. The entirety of China application No. 202111283975.6 is hereby incorporated by reference herein and made a part of this specification.

### TECHNICAL FIELD

The present application relates to the technical field of biomaterials for implant or intervention, and particularly to a total knee joint prosthesis for revision after a unicompartmental knee arthroplasty.

### BACKGROUND ART

A single condylar knee joint prosthesis is a surgical implant for replacing a knee joint and applied to a unicompartmental knee arthroplasty. An upper portion of the tibia and lower portion of the femur of human body need to be removed a half during the unicompartmental knee arthroplasty, and the half is used to accommodate the single condyle knee joint prosthesis.

However, when the single condylar knee joint is revised and replaced with a total knee joint replacement, the other half of the upper portion of the tibia and lower portion of the femur of human body need to be removed, and a tibia plateau after this removal and a tibia plateau after unicompartmental knee arthroplasty needs to be flush. Currently, a method of keeping flush between a tibia plateau after this removal and a tibia plateau after unicompartmental knee arthroplasty is that: a patch is increased at the tibia plateau after unicompartmental knee arthroplasty, then the patch is fixed on the top end of the human tibia through a locking nail, and the total knee joint prosthesis is mounted on the tibia plateau after this removal and the patch.

However, when the patch is fixed on the human tibia through the locking nail, it will additionally increase the pain of patient and results in a long operation time.

### SUMMARY

In order to reduce the pain of patient for revision after the unicompartmental knee arthroplasty and shorten the operation time, the present application provides a total knee joint prosthesis for revision after a unicompartmental knee arthroplasty.

The total knee joint prosthesis for revision after a unicompartmental knee arthroplasty in the present application adopts the following technical solution:

a total knee joint prosthesis for revision after a unicompartmental knee arthroplasty includes a tibial plateau and a tibial pad mounted on the tibial plateau, in which the tibial plateau includes an installation platform and an installation column fixedly connect to a side of the installation platform; a filling block is provided on a side of the installation platform towards the installation column; and the filling block is configured to be placed at a side of the human tibia where a unicompartmental knee arthroplasty is performed.

In the above technical solution, when the single condylar knee joint for revision is replaced by a total knee joint, an upper side of the tibia and a lower side of the femur of the human body may be directly treated according to requirement of surgery; then the tibial plateau is mounted on an upper side of the human tibia. Because the filling block is directly provided on the installation platform, a patch is not needed to be mounted through surgery on the side of the human tibia where the previous unicompartmental knee arthroplasty is performed, and the installation column is directly mounted on the upper side of the human tibia. At this time, the filling block is attached to the side of the human tibia where a unicompartmental knee arthroplasty is performed, which may reduce an additional pain of patient due to mounting the patch, and reduce operation steps of mounting the patch, and shorten the operation time.

In some embodiments, two limiting bosses are fixedly connected to a side of the installation platform away from the installation column; the two limiting bosses are separately located at two opposite sides of the installation platform; each of the two limiting bosses has an inclined side wall at a side facing with each other; each of the two limiting boss has a width configured to be gradually increased from a side close to the installation platform to a side away from the installation platform; and two chutes are formed on the tibial pad for the two limiting bosses to slide into.

In the above technical solution, the position of the tibial pad in the width direction of the limiting boss may be fixed by the cooperation of the two limiting bosses, avoiding the tibial pad from sliding along the width direction of the limiting bosses. Meanwhile, because each of the two limiting bosses has an inclined side wall at a side facing with each other, the position of the tibial pad in the axial direction may be limited.

In some embodiments, a front boss is fixedly connected to the installation platform, and a triangular structure is spatially formed by the front boss and the two limiting bosses.

In the above technical solution, the position of the tibial pad in the length direction of the limiting boss may be fixed by the front boss, avoiding the tibial pad from sliding on the installation platform under external force.

In some embodiments, a clamping slot is formed on a side of the front boss towards the limiting boss, and a clamping block configured to be snapped into the clamping slot is fixedly connected to a side of the tibial pad.

In the above technical solution, when the tibial pad is mounted on the installation platform, the clamping block on the tibial pad is exactly snapped into the clamping slot, so that a circumferential position of the tibial pad may be fixed, and the tibial pad may be fixed more firmly.

In some embodiments, the clamping block includes a fixing block fixedly connected to the tibial pad and an abutting block vertically and fixedly connected to the fixing block.

In the above technical solution, the position of the abutting block on the tibial pad may be fixed by the fixing block, and the abutting block may be directly snapped into the clamping slot, so that the front boss may be fixed with the tibial pad.

In some embodiments, a side of the abutting block away from the tibial pad is provided with a guiding surface.

In the above technical solution, the guiding surface can make the abutting block smoothly slide into the clamping slot, so that the front boss may be fixed with the tibial pad.

In some embodiments, a reinforcing rib is fixedly connected between the installation column and the installation platform.

In the above technical solution, the reinforcing rib can increase a connection strength between the installation platform and the installation column and reduce a possibility of fracture caused by the force between the two.

In some embodiments, a plurality of the reinforcing ribs are provided around a axis of the installation column at intervals.

In the above technical solution, the structural strength between the installation platform and the installation column is higher.

In some embodiments, a cement tank is formed on a side of the installation platform towards the installation column.

In the above technical solution, the cement tank may be filled with bone cement, the tibial plateau can not only be fixed on the human tibia through the installation column, but also through the bone cement, thereby increasing the contact area between the tibial plateau and the human tibia, so that the tibial plateau is fixed more firmly.

In summary, the present application has at least one of the following beneficial technical effects:

1. in the present application, a filling block is provided on a side of the installation platform towards the installation column; and the filling block is configured to be placed at a side of the human tibia where a unicompartmental knee arthroplasty is performed. Because the filling block is directly provided on the installation platform, a patch is not needed to be mounted through surgery on the side of the human tibia where the previous unicompartmental knee arthroplasty is performed, and the installation column is directly mounted on the upper side of the human tibia. At this time, the filling block is attached to the side of the human tibia where a unicompartmental knee arthroplasty is performed, which may reduce an additional pain of patient due to mounting the patch, and reduce operation steps of mounting the patch, and shorten the operation time;

2. a gradually changing width of the limiting boss may fix the position of the tibial pad in radial and axial direction at the same, and reduce the probability of the tibial pad sliding out from the holding chamber; and

3. the cement tank may increase the mounting area of the installation platform and the human tibia, so the tibial plateau is mounted more firmly, and the mounting stability is better.

### BRIEF DESCRIPTION OF THE DRAWINGS

FIG. 1 shows an overall structure diagram of the present application.

FIG. 2 shows a structure diagram of another perspective of the present application.

FIG. 3 shows a diagram illustrating a structure of the tibial plateau.

FIG. 4 shows a diagram illustrating a structure of the tibial pad.

### DETAILED DESCRIPTION

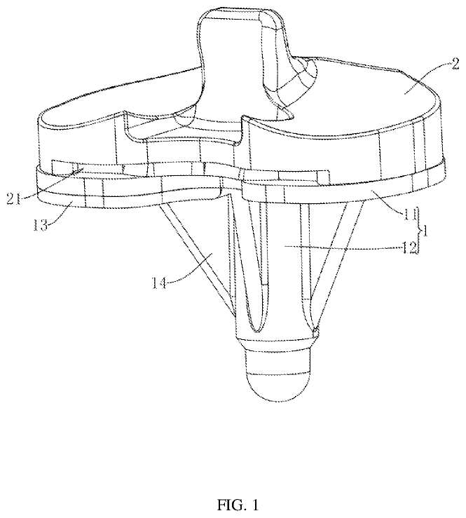

The present application is further described in detail below in combination with FIGS. 1-4.

An embodiment of the present application discloses a total knee joint prosthesis for revision after a unicompartmental knee arthroplasty. Referring to FIG. 1, the total knee joint prosthesis includes a tibial plateau 1 and a tibial pad 2 mounted on the tibial plateau 1, in which, the tibial plateau 1 includes an installation platform 11 and an installation column 12 fixedly connected to a side of the installation platform 11; a filling block 13 for increasing a thickness of the installation platform 11 is provided on a side of the installation platform 11 towards the installation column 12; the filling block 13 is configured to be placed at a side of a human tibia where a unicompartmental knee arthroplasty is performed; and the tibial pad is positioned at a side of the installation platform 11 away from the installation column 12. When the single condyle knee joint prosthesis is revised after a unicompartmental knee arthroplasty, the removed human tibia is more during a unicompartmental knee arthroplasty. Therefore, when the tibial plateau 1 is mounted during a total knee joint replacement surgery, the filling block 13 can be placed towards the side of the human tibia where a unicompartmental knee arthroplasty is performed; then the tibial plateau 1 is mounted on the human tibia; and the filling block 13 can be attached to the human tibia after unicompartmental knee arthroplasty. Therefore, the tibial plateau 1 may be mounted without adding a patch, and further the mounted tibial plateau 1 can be attached to the human tibia well. The operation time and operation steps for mounting a patch can be omitted; the pain of the patient caused by mounting a patch may be avoided; the operation steps may be simplified; and a whole operation time may be shortened.

In particular, a thickness of the filling block 13 can be designed according to actual situation. When the unicompartmental knee arthroplasty is operated, the severity of the illness of each patient is different, so the sizes of removed tibias are different. Therefore, a suitable thickness of the filling block 13 can be chosen according to the sizes of removed tibias of different patients in actual application. Moreover, the position of the filling block 13 also need be determined according to the position of the previous unicompartmental knee arthroplasty of the patient, such that the filling block 13 can be positioned at the side of the human tibia where a unicompartmental knee arthroplasty is preformed, that is, the filling block 13 will be positioned at the side where a patch is to be mounted. In the present application, the filling block 13 is fixedly connected to the installation platform 11, in which the operation is more convenient, the hurt to human body is reduced and the operation time is shortened, compared with the current operation of mounting a patch on the human tibia through a locking nail.

Meanwhile, the filling block 13 is fixedly connected to the installation platform 11. On one hand, such arrangement avoids the hurt to the human body caused by directly mounting the patch on the human tibia and shortens the operation time. On the other hand, a stepped surface is formed between a side of the filling block 13 towards the installation column 12 and the installation platform 11, which makes the installation platform 11 be attached to the human tibia better; plays a circumferential limiting role to the installation platform 11; and improves the stability of the installation platform 11 after installation.

In particular, in order to increase the connection strength between the installation platform 11 and installation column 12, a reinforcing rib 14 is fixedly connected between the installation platform 11 and the installation column 12; a plurality of the reinforcing ribs 14 are provided around the axis of the installation column 12 at equal intervals; and four reinforcing ribs 14 are adopted in the present application as an example.

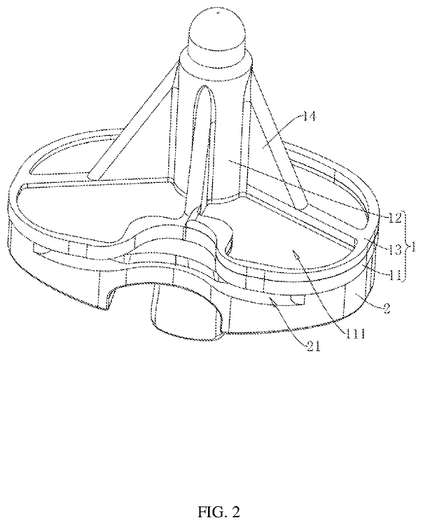

Referring to FIG. 2, when the tibial plateau 1 is mounted on the human tibia, the installation of the tibial plateau 1 may be achieved by fixedly connecting the installation column 12 to the human tibia. In particular, in order to increase the installation strength of the tibial plateau 1, a cement tank 111 is formed on a side of the installation platform 11 towards the installation column 12, and the cement tank 111 may be filled with bone cement. The tibial plateau 1 can be fixedly connected to the human tibia not only through the installation column 12, but also through the bone cement filled into the cement tank 111, so that the installation strength of the tibial plateau 1 can be more firmly.

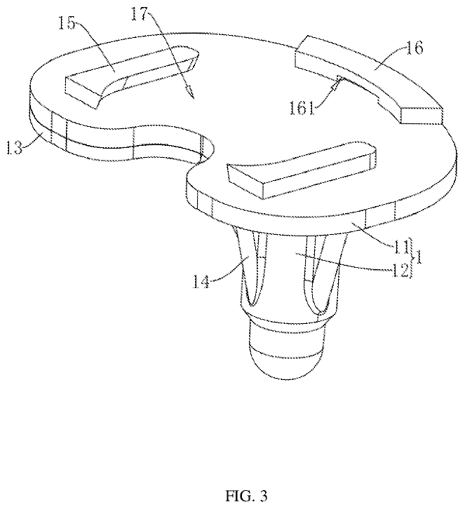

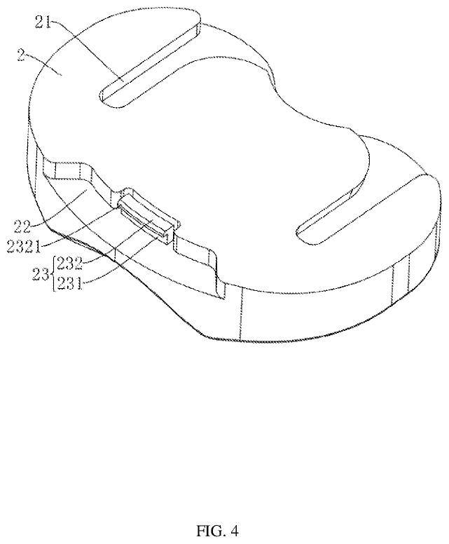

Referring to FIGS. 1 and 3, the tibial pad 2 is detachedly connected to a side of installation platform 11 away from the installation column 12. Specifically, two limiting bosses 15 are fixedly connected to a side of the installation platform 11 away from the installation column 12; the two limiting bosses 15 are separately positioned at two opposite sides of the installation platform 11. Each of the two limiting bosses 15 has an inclined side wall. Specifically, each of the two limiting boss 15 has a width configured to be gradually increased from a side close to the installation platform 11 to a side away from the installation platform 11. In particular, each of the two limiting bosses 15 has an inclined side wall which may be arranged at a side close to each other or a side away from each other. Referring to FIG. 4, two chutes 21 are formed on the tibial pad 2 to be embedded by the two limiting bosses 15. The tibial pad 2 may be fixed on the installation platform 11 through sliding the limiting boss 15 from a side of the tibial pad 2 to the chute 21, which may avoid the tibial pad 2 from sliding in the width direction of the limiting boss 15 and in a direction away from the installation platform 11.

Referring to FIGS. 3-4, in order to avoid the tibial pad 2 from sliding along a length direction of the limiting boss 15 at the installation platform 11 after the tibial pad 2 is mounted on the installation platform 11, a front boss 16 is fixedly connected to the installation platform 11, and the tibial pad 2 is formed with a holding groove 22 for embedding the front boss 16 to be embedded. A triangular structure is spatially formed by the front boss 16 and the two limiting bosses 15. And a holding chamber 17 for holding the tibial pad 2 is formed between the front boss 16 and the two limiting bosses 15. The tibial pad 2 is directly placed on the installation platform 11 from the holding chamber 17 during installation, then the tibial pad 2 is operated to slide towards the limiting bosses 15, such that the limiting bosses 15 is cooperated with the chute 21. The tibial pad 2 may be fixed in a radial direction and axial direction by the cooperation of the limiting bosses 15 and front boss 16. Therefore, the probabilities of the tibial pad 2 sliding on the tibial plateau 1 and the tibial pad 2 falling off the installation platform 11 can be reduced.

The position of the tibial pad 2 may be firmly fixed on the installation platform 11 when the tibial pad 2 is mounted on the installation platform 11 due to the arrangement of the chute 21 and holding groove 22. And the limiting boss 15 and the front boss 16 would not be exposed to the outside to affect the normal operation.

Referring to FIGS. 3-4, meanwhile, in order to limit a circumferential position of the tibial pad 2 and reduce the turning of the tibial pad 2 under an external force after being mounted on the installation platform 11, a clamping slot 161 is formed at a side of the front boss 16 towards the limiting bosses 15, and a clamping block 23 configured to be snapped into the clamping slot 161 is fixedly connected to the tibial pad 2 and also fixedly connected to a sidewall of the holding groove 22. The clamping block 23 includes a fixing block 231 fixedly connected to the tibial pad 2 and a abutting block 232 vertically and fixedly connected to a side of the fixing block 231. The abutting block 232 and the fixing block 231 are integrally formed, so that the abutting block 232 and the fixing block 231 can form an elastic part. When the tibial pad 2 is slid from the holding chamber 17 to the limiting bosses 15, the two limiting bosses 15 can cooperate with the chute 21; then the tibial pad 2 is directly pressed down; the abutting block 232 will abut against a sidewall of the front boss 16 and is elastically moved relative to the tibial pad 2 under the counterforce of the front boss 16 until the abutting block 232 slides into the clamping slot 161, thereby the circumferential position of the tibial pad 2 is fixed. Further, in order to facilitate the sliding of the holding groove 22 into the clamping slot 161, a guiding surface 2321 is provided at a side of the abutting block 232 abutted against the front boss 16, which is positioned at a side of the abutting block 232 away from the fixing block 231. When the tibial pad 2 is placed on the installation platform 11 from the holding chamber 17, the two limiting bosses 15 will cooperate with the chute 21; then the tibial pad 2 is pressed; the guiding surface 2321 of the abutting block 232 will abut against the front boss 16 and smoothly slide into the clamping slot 161; and then the circumferential position of the tibial pad 2 is fixed.

An implementation principle of a total knee joint prosthesis for revision after a unicompartmental knee arthroplasty in an embodiment of the present application is that: when the single condylar knee joint prosthesis is replaced by a total knee joint replacement, the filling block 13 may be arranged according to the position and size of the patch needed to be added, and the filling block 13 may be fixedly connected to the installation platform 11. During the operation, the doctor may directly insert the installation column 12 into a position of the human tibia to be mounted, therefore a stepped surface of the installation platform 11 towards a side of the installation column 12 is cooperated with a stepped surface of the top end of the human tibia cut in the unicompartmental knee arthroplasty. The doctor can directly operate the total knee joint replacement surgery, and need not to mount the patch on the human tibia through the locking nail, thereby reducing the pain of the patient during revision, reducing the operation step and shortening the operation time. During the operation, the installation column 12 can be directly fixed on the human tibia by the bone cement, and the installation platform 11 can be fixed on the human tibia through bone cement filled into the cement tank 111. Then, the tibial pad 2 is mounted on the installation platform 11, so the tibial pad 2 may be fixed on the tibial plateau 1.

The above are the preferred embodiments of the present application, which are not intended to limit the protection scope of the present application. Therefore, all equivalent changes made according to the structure, shape and principle of the present application should be covered within the protection scope of the present application.

What is claimed is:

1. A total knee joint prosthesis for revision after a unicompartmental knee arthroplasty, comprising a tibial plateau and a tibial pad mounted on the tibial plateau, wherein, the tibial plateau comprises an installation platform and an installation column fixedly connected to a side of the installation platform; a filling block is provided on the side of the installation platform towards the installation column; and the filling block is configured to be placed at a side of a human tibia where the unicompartmental knee arthroplasty is performed.

2. The total knee joint prosthesis for revision after a unicompartmental knee arthroplasty according to claim 1, wherein, two limiting bosses are fixedly connected to a second side of the installation platform away from the installation column; the two limiting bosses are separately located at two opposite sides of the installation platform; each of the two limiting bosses has an inclined side wall; each of the two limiting boss has a width configured to be gradually increased from a side close to the installation platform to a side away from the installation platform; and two chutes are formed on the tibial pad for the two limiting bosses to slide into.

3. The total knee joint prosthesis for revision after a unicompartmental knee arthroplasty according to claim 2, wherein, a front boss is fixedly connected to the installation platform; and a triangular structure is spatially formed by the front boss and the two limiting bosses.

4. The total knee joint prosthesis for revision after a unicompartmental knee arthroplasty according to claim 3, wherein, a clamping slot is formed on a side of the front boss towards the two limiting bosses; and a clamping block configured to be snapped into the clamping slot is fixedly connected to a side of the tibial pad.

5. The total knee joint prosthesis for revision after a unicompartmental knee arthroplasty according to claim 4, wherein, the clamping block comprises a fixing block fixedly connected to the tibial pad and an abutting block vertically and fixedly connected to the fixing block.

6. The total knee joint prosthesis for revision after a unicompartmental knee arthroplasty according to claim 5, wherein, a side of the abutting block away from the tibial pad is provided with a guiding surface.

7. The total knee joint prosthesis for revision after a unicompartmental knee arthroplasty according to claim 1, wherein, a reinforcing rib is fixedly connected between the installation column and the installation platform.

8. The total knee joint prosthesis for revision after a unicompartmental knee arthroplasty according to claim 7, wherein, a plurality of the reinforcing ribs are provided around an axis of the installation column at intervals.

9. The total knee joint prosthesis for revision after a unicompartmental knee arthroplasty according to claim 1, wherein, a cement tank is formed on the side of the installation platform towards the installation column.
